(19) United States
(12) Patent Application Publication
(10) Pub. No.: US 2015/0164459 A1
(43) Pub. Date: Jun. 18, 2015

(54) COMMUNICATION CONTROL METHOD AND RADIOGRAPHIC IMAGING APPARATUS AND SYSTEM

(71) Applicant: FUJIFILM Corporation, Tokyo (JP)

(72) Inventor: Takaaki ITO, Ashigarakami-gun (JP)

(73) Assignee: FUJIFILM CORPORATION, Tokyo (JP)

(21) Appl. No.: 14/634,303

(22) Filed: Feb. 27, 2015

Related U.S. Application Data
(63) Continuation of application No. PCT/JP2013/072970, filed on Aug. 28, 2013.

(30) Foreign Application Priority Data
Aug. 31, 2012 (JP) 2012-191130

(51) Int. Cl. A61B 6/00

(52) U.S. Cl. CPC A61B 6/542; A61B 6/563; A61B 6/405

(57) ABSTRACT

An X-ray imaging apparatus includes a detection panel for receiving X-rays transmitted through an object after emission from an X-ray source, and converting an X-ray image of the object into an electric signal. An AEC signal output device detects a dose of the X-rays and outputs an AEC signal for exposure control of the X-ray image. A sync communication interface establishes a first communication path for transmitting a sync signal to the radiation source controller for controlling the X-ray source, for driving the detection panel in synchronism with a start of the irradiation. There is an AEC interface for coupling with an AEC interface provided in the radiation source controller for connection with a second AEC signal output device discrete from the AEC signal output device, and for establishing a second communication path for transmitting an AEC signal to the radiation source controller.

### CROSS-REFERENCE TO RELATED APPLICATION

This application is a Continuation of PCT International Application PCT/JP2013/072970 filed on 28 Aug. 2013, which claims priority under 35 USC 119(a) from Japanese Patent Application No. 2012-191130 filed on 31 Aug. 2012. The above application is hereby expressly incorporated by reference, in its entirety, into the present application.

### BACKGROUND OF THE INVENTION

1. Field of the Invention

The present invention relates to a communication control method and radiographic imaging apparatus and system. More particularly, the present invention relates to a communication control method and radiographic imaging apparatus and system, in which a radiographic imaging apparatus with an AEC function (automatic exposure control function) can be utilized in combination with a radiation source controller easily without excessive complication of a structure.

2. Description Related to the Prior Art

An X-ray imaging system is well-known in the medical field in which X-rays are used as radiation. The X-ray imaging system includes an X-ray generating apparatus and an X-ray imaging apparatus. The X-ray generating apparatus generates X-rays. The X-ray imaging apparatus images a body or object receiving X-rays, to form an X-ray image. The X-ray generating apparatus includes an X-ray source, a radiation source controller, and a start switch. The X-ray source emits X-rays toward the body of a patient. The radiation source controller controls operation of the X-ray source. The start switch inputs a start signal to the radiation source controller for irradiation. The X-ray imaging apparatus includes a radiographic imaging device (X-ray imaging device), and a console unit. The radiographic imaging device detects the X-ray image according to the X-rays passed through the body. The console unit controls the radiographic imaging device, and stores and displays the X-ray image.

A recently available type of the X-ray imaging device electronically detects an X-ray image instead of using an X-ray film, IP cassette or the like. The radiographic imaging device includes a detection panel (sensor panel) and a signal processor. The detection panel, for example, flat panel detector (FPD), detects an X-ray image as electric signal. Pixels are arranged on the detection panel two-dimensionally for storing signal charge according to a dose of X-rays. The signal processor has a switching element. The signal charge is read out from pixels through the switching element, for example, TFT (thin film transistor), and stored, and converted into a voltage signal. The signal processor outputs an image signal for constituting the X-ray image.

In the X-ray imaging apparatus, the sensor panel periodically resets unwanted charge of pixels before radiographic imaging in a manner unlike the imaging with the X-ray film or IP cassette, for the purpose of abandoning electric noise of dark current charge in an X-ray image. It is necessary to synchronize a first time point of the sensor panel with a second time point of the radiation source controller. At the first time point, the sensor panel stops the pixel reset and starts storing signal charge in pixels. At the second time point, X-rays start being emitted.

Assuming that the storing is continued even after the stop of the X-ray irradiation, the image quality of an X-ray image will be lowered by electric noise of dark current charge in the signal charge. Thus, it is also necessary to synchronize a time point of starting the readout of the signal charge in the signal processor upon the stop of the storing with a time point of the stop of the X-ray irradiation.

JP-A 2011-041866 discloses synchronization of the start and stop in an X-ray imaging system having an X-ray imaging apparatus. In this document, the radiation source controller is connected with the X-ray imaging apparatus communicably by a network connection board as an accessory of the radiation source controller. A single signal line connects the radiation source controller to the network connection board. A communication path between the network connection board and the X-ray imaging apparatus is a combined structure of wireless communication and wired communication. Each of the radiation source controller and the X-ray imaging apparatus is provided with one interface for transmitting a sync signal for the start and a sync signal for the stop. Thus, one communication path connects the radiation source controller with the X-ray imaging apparatus.

In FIG. 14, steps of synchronizing the start of the X-ray imaging system having the X-ray imaging apparatus of JP-A 2011-041866 are illustrated. At first, a radiation source controller 200 (source driver) transmits a request signal S1 as a sync signal to an X-ray imaging apparatus 201 as radiographic imaging apparatus, to request allowance of a start of X-ray irradiation. The X-ray imaging apparatus 201 in response to the request signal S1 stops the pixel reset and starts the storing, and also transmits an enable signal S2 to the radiation source controller 200 for enabling the X-ray irradiation as a sync signal. The radiation source controller 200 starts the X-ray irradiation in response to the enable signal S2. For steps of synchronizing the stop of the X-ray imaging system, the radiation source controller 200 transmits an end flag S3 as a sync signal to the X-ray imaging apparatus 201, to notify a stop of X-ray irradiation upon the end of the X-ray irradiation at the radiation source controller 200. The X-ray imaging apparatus 201 in response to the end flag S3 changes over from the storing to the readout. One communication path 202 or signal path is used for transmitting and receiving sync signals between the radiation source controller 200 and the X-ray imaging apparatus 201 for start and stop.

An AEC function (automatic exposure control function) is used in the X-ray imaging system, in which dose of X-rays is detected by a monitoring sensor, to stop irradiation of X-rays upon reach of a cumulative dose of the dose to a target dose, so as to obtain a radiation image of a high quality and preventing overexposure of radiation to a body or object. The dose depends upon a current-time product (mAs value) as a product of irradiation time of X-rays and a tube current as a factor to determine the dose per unit time. An imaging condition including the irradiation time and the tube current is predetermined as a recommendable condition according to a body part (chest, head and the like), sex, age and other specification of the patient (body). However, a transmission coefficient of X-rays varies according to specificity of the body, for example, a body size. Thus, the AEC is performed for obtaining higher image quality.

For the purpose of the AEC, an AEC signal output device (radiation monitoring device) is used, in which a monitoring sensor outputs a dose signal of a dose per unit time as an AEC signal (monitoring signal). Conventionally, an AEC signal output device separate from the X-ray imaging apparatus is used, for example, an ionization chamber. There has been a recent type of an X-ray imaging apparatus including a built-in AEC signal output device for the AEC. The AEC signal output device has the monitoring sensor in a similar manner to the separate type of the AEC signal output device. Also, the AEC signal output device includes an evaluator for determining a cumulative dose according to the dose signal and checking whether the cumulative dose has becomes as high as the target dose. Examples of the AEC signal output by the AEC signal output device to a radiation source controller are the dose signal, a stop signal according to a result of evaluation from the evaluator, and the like. For this purpose, a radiation source controller has a function for receiving the AEC signal.

For example, JP-A 2011-153876 discloses an X-ray imaging apparatus having a monitoring sensor and an evaluator. The monitoring sensor detects a dose of radiation according to a current flowing in a cable line which interconnects a driver and a power source circuit, the driver turning on and off switching elements in the sensor panel, the power source circuit supplying the driver with drive voltage. The evaluator checks whether a cumulative dose of the dose has become as high as a target dose. In a similar manner to JP-A 2011-041866, each of the radiation source controller and the X-ray imaging apparatus has one interface for communication of a sync signal and an AEC signal in JP-A 2011-153876. One communication path connects the radiation source controller to the X-ray imaging apparatus.

Steps of synchronizing the start in an X-ray imaging system having an X-ray imaging apparatus 211 as radiographic imaging apparatus with an AEC by a monitoring sensor in FIG. 15 are the same as those in an X-ray imaging system having the X-ray imaging apparatus 201 without an AEC in JP-A 2011-041866. For steps of synchronizing the stop, the X-ray imaging apparatus 211 starts sampling an output (dose signal S4) of the monitoring sensor upon the start of the storing of the X-ray imaging apparatus 211. The dose signal S4 as an AEC signal is transmitted to a radiation source controller 210 (source driver) by the X-ray imaging apparatus 211. The evaluator in the radiation source controller 210 in response to the dose signal S4 checks whether the cumulative dose has become as high as the target dose according to a cumulative value of the dose signal. In case it is judged that the cumulative dose has become as high as the target dose, the radiation source controller 210 stops the X-ray irradiation, and transmits the end flag S3 to the X-ray imaging apparatus 211. The X-ray imaging apparatus 211 changes over from the storing to the readout in response to the end flag S3. In a similar manner to the example of FIG. 14, one communication path 212 or signal path is used for transmitting a sync signal for the start and a sync signal for the stop between the radiation source controller 210 and the X-ray imaging apparatus 211, and transmitting and receiving the AEC signal.

In FIG. 16, an X-ray imaging apparatus 221 as radiographic imaging apparatus of JP-A 2011-153876 includes the monitoring sensor and the evaluator. In case the evaluator detects the reach of the cumulative dose to the target dose, the X-ray imaging apparatus 221 changes over from the storing to the readout. Also, a stop signal S5 as an AEC signal is transmitted to a radiation source controller 220 (source driver) by the X-ray imaging apparatus 221 for stopping the irradiation. The radiation source controller 220 stops the irradiation in response to the stop signal S5. In a similar manner to FIGS. 14 and 15, one communication path 222 or signal path is used for transmitting the sync signal between the radiation source controller 220 and the X-ray imaging apparatus 221 and transmitting and receiving the AEC signal.

Assuming that an X-ray imaging system is constructed by an X-ray generating apparatus and an X-ray imaging apparatus of the same manufacturer, it is possible with high degree of freedom to determine specifics of the interfaces between a radiation source controller and the X-ray imaging apparatus and forms of signals transmitted through the interfaces. Accordingly, it is preferable to minimize the number of parts constituting a communication path between the radiation source controller and the X-ray imaging apparatus in consideration of handlability of devices and labor for their installation. The communication path is normally single between the radiation source controller and the X-ray imaging apparatus as suggested in JP-A 2011-041866 and JP-A 2011-153876.

In one hospital facility, the X-ray imaging apparatus 201 of FIG. 14 without the AEC function of a monitoring sensor or evaluator is installed. It is conceivable in the hospital facility to introduce the X-ray imaging apparatus 211 or 221 (FIGS. 15 and 16) having the AEC function. Assuming that an X-ray generating apparatus is exchanged entirely for renewal in compliance with the X-ray imaging apparatus 211 or 221 with the AEC, the expense for the renewal will be excessively high. Thus, the reuse of the X-ray generating apparatus installed in the hospital facility is desired. Note that there is no problem of synchronizing the start (communication of the request signal S1 and enable signal S2), because there is no difference between the X-ray imaging apparatus 201 and the X-ray imaging apparatus 211 or 221.

However, the radiation source controller 200 does not have a particular function for receiving the AEC signal (dose signal S4 or stop signal S5) through the communication path 202 in contrast with a function for transmitting and receiving sync signals for the start and stop through the communication path 202, because no use in combination with the X-ray imaging apparatus 211 or 221 with the AEC is considered. Assuming that connection of the radiation source controller 200 to the X-ray imaging apparatus 211 or 221 with the communication path 202 is carried out as illustrated in FIG. 17 in the manner of the known techniques, the radiation source controller 200 is required to have a processing circuit, program or the like for receiving the AEC signal, specifically, for recognizing the AEC signal or effectively stopping the X-ray irradiation upon receiving the AEC signal. This will increase the cost and labor for the structural modification.

In case the X-ray imaging apparatus having the AEC function is introduced into the X-ray imaging system including an X-ray imaging apparatus with an X-ray film or IP cassette, it is impossible to transmit a sync signal for the start, a sync signal for the stop, and the AEC signal by use of one communication path. Modification of the X-ray imaging system requires a high cost and much labor.

### SUMMARY OF THE INVENTION

In view of the foregoing problems, an object of the present invention is to provide a communication control method and radiographic imaging apparatus and system, in which a radiographic imaging apparatus with an AEC function (automatic exposure control function) can be utilized in combination with a radiation source controller easily without excessive complication of a structure.

In order to achieve the above and other objects and advantages of this invention, a communication control method for a radiographic imaging system is provided, the radiographic imaging system including a radiation generating apparatus, having a radiation source for emitting radiation, and a radiation source controller for controlling the radiation source, and a radiographic imaging apparatus, having a detection panel for receiving the radiation transmitted through an object and converting a radiation image of the object into an electric signal, and an AEC signal output device for detecting a dose of the radiation and outputting an AEC signal for exposure control of the radiation image. The communication control method includes a step of transmitting a sync signal through a first communication path between the radiation source controller and the radiographic imaging apparatus, for driving the detection panel in synchronism with a start of the irradiation of the radiation. The AEC signal is transmitted from the radiographic imaging apparatus to the radiation source controller through a second communication path between the radiation source controller and the radiographic imaging apparatus, by use of an AEC interface provided in the radiation source controller for connection with an AEC signal output device discrete from the radiographic imaging apparatus.

Preferably, the AEC signal is transmitted in a form receivable by the AEC interface.

Preferably, the radiographic imaging apparatus outputs the AEC signal in a form receivable by the AEC interface.

Preferably, the AEC signal from the radiographic imaging apparatus is converted by a signal converter in the second communication path into a form receivable by the AEC interface.

Preferably, the second communication path is at least partially wired with a cable line.

Preferably, a branch connector is provided in the cable line, and has first, second and third terminals. The first terminal is coupled with the AEC interface, the second terminal is coupled with the radiographic imaging apparatus, and the third terminal is coupled with the AEC signal output device.

Preferably, the AEC signal output device and the radiographic imaging apparatus are changed over selectively in the branch connector for transmission of the AEC signal to the AEC interface.

In another preferred embodiment, the second communication path is at least partially wireless.

Preferably, the AEC signal is output wirelessly by the radiographic imaging apparatus, then converted into a wired signal, and input to the AEC interface.

Preferably, the first communication path is established by a sync communication interface provided in the radiation source controller for transmitting the sync signal.

In still another preferred embodiment, a radiation switch is connected to the radiation source controller, and generates a signal for starting irradiation of the radiation, to establish the first communication path.

Preferably, the first communication path is established by a signal routing device connected to the radiation switch, the radiation generating apparatus and the radiographic imaging apparatus.

Preferably, the radiographic imaging apparatus includes a sync communication interface for establishing the first communication path. A second AEC interface establishes the second communication path.

In a further preferred embodiment, the radiographic imaging apparatus includes a common interface for communication of the sync signal and the AEC signal. A first cable line is connected to the radiation source controller, for transmitting the sync signal. A second cable line is connected to the AEC interface, for transmitting the AEC signal. The first and second cable lines are coupled together and connected to a third cable line, the third cable line is connected to the common interface, for establishing the first and the second communication paths.

Preferably, the AEC signal output device includes a monitoring sensor for detecting the dose of the radiation. The AEC signal is a dose signal from the monitoring sensor. The radiation source controller includes an evaluator for checking whether a cumulative dose has become equal to a target dose according to the dose signal.

Preferably, the AEC signal output device includes a monitoring sensor for detecting the dose of the radiation. An evaluator checks whether a cumulative dose has become equal to a target dose according to a dose signal from the monitoring sensor. Assuming that the evaluator judges that the cumulative dose has become equal to the target dose, a stop signal is output for the AEC signal to stop the radiation source controller from irradiation.

Also, a radiographic imaging system is provided, and includes a radiation generating apparatus, having a radiation source for emitting radiation, and a radiation source controller for controlling the radiation source. A radiographic imaging apparatus has a detection panel for receiving the radiation transmitted through an object and converting a radiation image of the object into an electric signal, and an AEC signal output device for detecting a dose of the radiation and outputting an AEC signal for exposure control of the radiation image. A first communication path is established between the radiation source controller and the radiographic imaging apparatus, for transmitting a sync signal for driving the detection panel in synchronism with a start of the irradiation of the radiation. A second communication path is established between the radiation source controller and the radiographic imaging apparatus by use of an AEC interface provided in the radiation source controller for connection with an AEC signal output device discrete from the radiographic imaging apparatus, for transmitting the AEC signal from the radiographic imaging apparatus to the radiation source controller.

Also, a radiographic imaging apparatus includes a detection panel for receiving radiation transmitted through an object after emission from a radiation source, and converting a radiation image of the object into an electric signal. An AEC signal output device detects a dose of the radiation and outputs an AEC signal for exposure control of the radiation image. A sync communication interface establishes a first communication path for transmitting a sync signal to a radiation source controller for controlling the radiation source, for driving the detection panel in synchronism with a start of the irradiation of the radiation. There is an AEC interface for coupling with a second AEC interface provided in the radiation source controller for connection with an AEC signal output device discrete from the AEC signal output device, and for establishing a second communication path for transmitting the AEC signal to the radiation source controller.

Thus, a radiographic imaging apparatus with an AEC function (automatic exposure control function) can be utilized in combination with a radiation source controller easily without excessive complication of a structure, because the first and second communication paths can be utilized separately between the sync signal and the AEC signal to be transmitted.

### BRIEF DESCRIPTION OF THE DRAWINGS

The above objects and advantages of the present invention will become more apparent from the following detailed description when read in connection with the accompanying drawings, in which:

FIG. 1 is a block diagram schematically illustrating an X-ray imaging system;

FIG. 2 is a block diagram schematically illustrating the X-ray imaging system;

FIG. 3 is a block diagram schematically illustrating first and second communication paths;

FIG. 4 is a flow chart illustrating steps preparatory for X-ray imaging;

FIG. 5 is a flow chart illustrating the X-ray imaging;

FIG. 6 is a block diagram schematically illustrating a preferred embodiment having a signal routing device;

FIG. 7 is a block diagram schematically illustrating an embodiment in which a cable line from an ionization chamber is coupled with a cable line from an electronic cassette;

FIG. 8 is a block diagram schematically illustrating an embodiment in which a selector is provided between cable lines from the ionization chamber and the electronic cassette;

FIG. 9 is a block diagram schematically illustrating an embodiment in which first and second communication paths are wired totally;

FIG. 10 is a block diagram schematically illustrating an embodiment in which first and second communication paths are wireless partially;

FIG. 11 is a block diagram schematically illustrating a preferred electronic cassette having an AEC function;

FIG. 12 is a block diagram schematically illustrating conversion of a signal from the electronic cassette into a form receivable by an AEC interface;

FIG. 13 is a block diagram schematically illustrating an electronic cassette having a common interface;

FIG. 14 is a block diagram schematically illustrating transmission of a signal between an X-ray imaging apparatus and a radiation source controller without an AEC;

FIG. 15 is a block diagram schematically illustrating transmission of a signal between an X-ray imaging apparatus and a radiation source controller with a monitoring sensor;

FIG. 16 is a block diagram schematically illustrating transmission of a signal between an X-ray imaging apparatus and a radiation source controller with a monitoring sensor and an evaluator;

FIG. 17 is a block diagram schematically illustrating transmission of a signal between an X-ray imaging apparatus with an AEC and a radiation source controller without compatibility to an AEC.

### DETAILED DESCRIPTION OF THE PREFERRED EMBODIMENT(S) OF THE PRESENT INVENTION

### First Embodiment

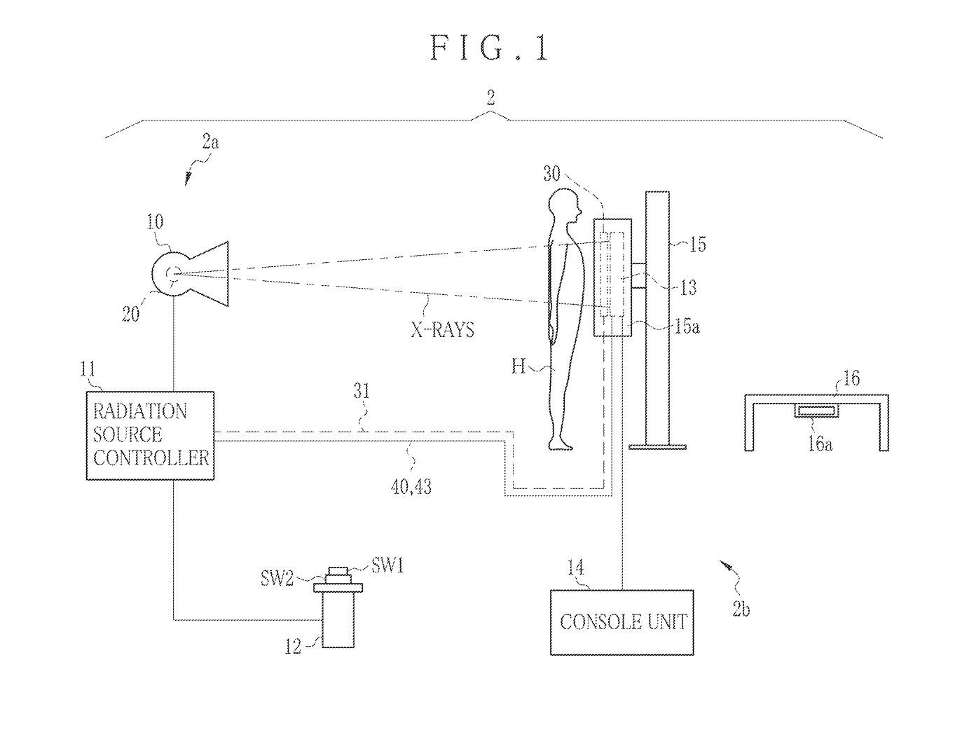

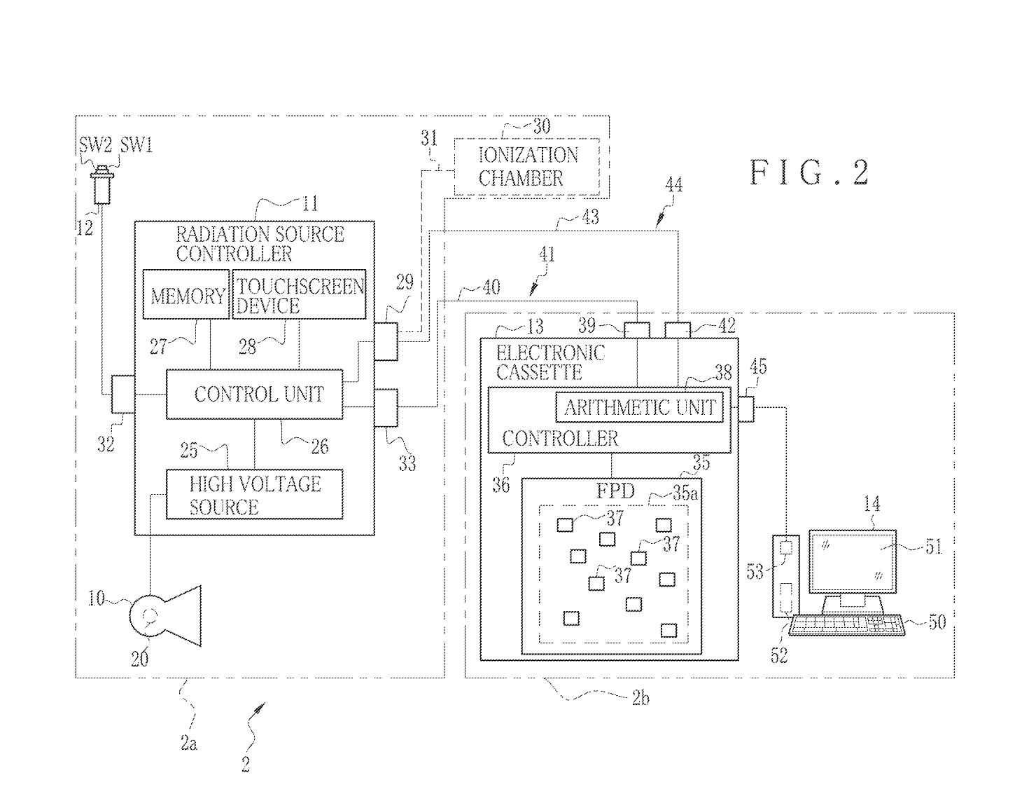

In FIGS. 1 and 2, an X-ray imaging system 2 as radiographic imaging system includes an X-ray source 10, a radiation source controller 11 (source driver), a radiation switch 12, an electronic cassette 13 as a radiographic imaging device, a console unit 14, a standing orientation imaging station 15 (floor stand) and a horizontal orientation imaging station 16 (imaging table). The radiation source controller 11 controls the X-ray source 10. The radiation switch 12 functions for starting warmup of the X-ray source 10 and starting irradiation of X-rays. The electronic cassette 13 outputs an X-ray image by detecting X-rays from a body H (object) of a patient. The console unit 14 controls operation of the electronic cassette 13 and display of the X-ray image. The standing orientation imaging station 15 is used for imaging the body H in a standing orientation. The horizontal orientation imaging station 16 is used for imaging the body H in a horizontal orientation. An X-ray generating apparatus 2a (radiation generating apparatus) is constituted by the X-ray source 10, the radiation source controller 11 and the radiation switch 12. An X-ray imaging apparatus 2b as radiographic imaging apparatus is constituted by the electronic cassette 13 and the console unit 14. A moving mechanism (not shown) is provided and sets the X-ray source 10 in a desired direction and position. The X-ray source 10 is commonly used with the imaging stations 15 and 16.

The X-ray source 10 includes an X-ray tube 20 and a collimator (not shown). The collimator limits a field of irradiation of X-rays from the X-ray tube 20. The X-ray tube 20 includes a negative electrode and a positive electrode. The negative electrode is a filament for emitting thermal electron. The positive electrode is a target receiving collision of the thermal electron, and emits X-rays. The collimator is constituted by four movable plates of metal lead arranged quadrilaterally for blocking X-rays. An emission opening of a quadrilateral shape is defined in the collimator at the center for transmitting X-rays. The movable plates are moved to adjust the size of the emission opening, to change the field of irradiation.

The radiation source controller 11 includes a high voltage source 25 and a control unit 26. The high voltage source 25 generates a tube voltage of a high level by boosting an input voltage with a transformer, and supplies the tube voltage to the X-ray tube 20. The control unit 26 controls the tube voltage to determine an energy spectrum of X-rays from the X-ray source 10, a tube current to determine dose per unit time, and irradiation time of X-rays. A high voltage cable connects the high voltage source 25 to the X-ray source 10.

A memory 27 stores a plurality of data of irradiation conditions, inclusive of the tube voltage, tube current, irradiation time, stop threshold and the like. The stop threshold is used for triggering a stop of X-ray irradiation in the control unit 26. The irradiation conditions are predetermined for plural body parts. There is a touchscreen device 28 or user input interface or input device, with which one of the irradiation conditions is manually set by a physician or operator, for example, radiologist or radiology technician. Also, information of a selected one of the film cassette, IP cassette and electronic cassette (one of the types with and without the AEC function) is selected by use of the touchscreen device 28. A countdown timer (not shown) is incorporated in the control unit 26 for stopping the irradiation of X-rays in case the measured time in the countdown timer becomes the irradiation time.

Irradiation time for the AEC is predetermined sufficiently long for the purpose of preventing shortage in the dose, because the irradiation of X-rays must not be stopped before detecting the reach to the target dose for stopping the irradiation in the AEC. An example of the irradiation time can be a maximum irradiation time in view of the safety regulation in the X-ray source 10. The control unit 26 performs control of the X-ray irradiation according to the tube voltage, tube current and irradiation time of the determined imaging condition. Assuming that it is judged that the cumulative dose of X-rays has come up to the target dose of sufficiency, the AEC functions to stop the X-ray irradiation even in case the measured time is equal to or less than the irradiation time set in the radiation source controller 11. Assuming that no AEC is used, irradiation time is set according to a body part to be imaged. The control unit 26 stops the X-ray irradiation upon the lapse of the set irradiation time in the internal countdown timer.

An ionization chamber 30 as a separate AEC signal output device (radiation monitoring device) is coupled to an AEC interface 29 (I/F) or monitor interface by a cable line 31 or wire line. An example of the AEC interface 29 is an electrical connector with a pair of metal contact plates for squeezing an end of a core line of the cable line 31 in which a jacket layer of the cable line 31 is locally peeled to uncover the line end. The AEC interface 29 can have other forms, for example, a connector of a plug-in structure.

The ionization chamber 30 is used for imaging with a film cassette or IP cassette, or an electronic cassette without an AEC. The imaging stations 15 and 16 have cassette holders 15a and 16a. The ionization chamber 30 is disposed in front of or behind a cassette at each of the cassette holders 15a and 16a. In FIG. 1, the ionization chamber 30 is set in the cassette holder 15a of the standing orientation imaging station 15. The ionization chamber 30 can be set on the cassette holder 16a of the horizontal orientation imaging station 16, and used commonly between the imaging stations 15 and 16. To use the electronic cassette 13 having the AEC, the ionization chamber 30 is removed from the AEC interface 29 as indicated by the broken line.

The ionization chamber 30 has a radiation receiving area for a predetermined body part, such as one of right and left lungs, a lower part of the center of the abdomen, and the like. The ionization chamber 30 outputs a voltage signal or first dose signal at a predetermined sampling interval according to a dose of X-rays incident upon the radiation receiving area after passage through the body H.

The ionization chamber 30 is a device of a type used widely in combination with a film cassette or IP cassette. The AEC interface 29 is provided in nearly every one of various types of the radiation source controller 11.

A switch interface 32 (I/F) or connection port connects the radiation switch 12 to the control unit 26. A physician or operator manipulates the radiation switch 12 for start of X-ray irradiation. Two switch buttons SW1 and SW2 are in a nested form in one another. The switch button SW2 can be turned on only after depressing the switch button SW1 in the double button structure of the radiation switch 12. The control unit 26 upon receiving an operation signal of halfway depression of the radiation switch 12 (turning on the switch button SW1) outputs a warmup signal for starting warming up the X-ray tube 20. The control unit 26 upon receiving an operation signal of full depression of the radiation switch 12 (turning on the switch button SW2) outputs one of a drive signal and a request signal S1 (corresponding to a sync signal). Assuming that the use of the film cassette or IP cassette is selected, the control unit 26 outputs the drive signal. Assuming that the use of the electronic cassette is selected, the control unit 26 outputs the request signal S1 for requesting start of the X-ray irradiation. A sync communication interface 33 (I/F) or synchronization interface transmits the request signal S1 to the electronic cassette. The control unit 26, upon receiving an enable signal S2 (corresponding to a sync signal) from the sync communication interface 33 as a response to the request signal S1, generates the drive signal for irradiation. The warmup signal and the drive signal are transmitted to the high voltage source 25. The high voltage source 25 starts supplying the X-ray tube 20 with power for irradiation upon receiving the drive signal from the control unit 26.

The electronic cassette 13 includes a detection panel 35 (FPD) or sensor panel, and a controller 36 for controlling the detection panel 35. The detection panel 35 has a plurality of arrays of pixels arranged on a TFT active matrix substrate. The pixels receive X-rays transmitted through the body H after emission from the X-ray source 10, and store charge. Each of the pixels includes a photoconductor, a capacitor, and a TFT (thin film transistor) as a switching element. The photoconductor generates charge (electron-hole pair) upon receiving visible light. The capacitor stores the charge generated by the photoconductor. The detection panel 35 reads out the signal charge stored in the photoconductor of the pixels through signal lines associated with pixel rows, so that the signal processing circuit converts the signal charge into a voltage signal to output an X-ray image. Note that the pixels may not have a capacitor.

The detection panel 35 includes scintillator for converting X-rays into visible light. The detection panel 35 is an indirect conversion type in which visible light output by the scintillator is converted photoelectrically by pixels into electric charge. It is possible to arrange the scintillator and the TFT active matrix substrate in the travel direction of X-rays, namely according to the arrangement of the penetration side sampling (PSS), or arrange the scintillator and the TFT active matrix substrate in a direction opposite to the travel direction of X-rays, namely according to the arrangement of the irradiation side sampling (ISS). Also, the scintillator may not be formed. It is possible to use a detection panel of a direct conversion type having a conversion layer of amorphous selenium or the like for directly converting X-rays into electric charge without the scintillator.

The controller 36 drives the TFT through scan lines associated with pixel rows, so that the detection panel 35 operates in pixel phases of storing, image readout and pixel reset. In the storing, the detection panel 35 causes the pixels to store signal charge according to a dose of X-rays. In the image readout, the signal charge is read out of the pixels. In the pixel reset, dark current charge is read out of the pixels for abandonment or resetting. The controller 36 performs image processing of various functions to an X-ray image output from the detection panel 35 by image readout, for example, offset correction, sensitivity correction, defect correction and the like.

The electronic cassette 13 is the radiographic imaging device including an AEC signal output device, which is constituted by monitoring sensors 37 or radiation monitoring device with the detection panel 35. An active pixel area 35a of the detection panel 35 includes numerous pixels. The monitoring sensors 37 detect dose of X-rays incident upon the active pixel area 35a, and outputs a second dose signal S4 (corresponding to an AEC signal or monitoring signal). An arithmetic unit 38 is supplied by the monitoring sensors 37 with the second dose signal S4. The monitoring sensors 37 are arranged discretely within the entire region of the active pixel area 35a without local deviation.

The monitoring sensors 37 are part of the pixels. Monitor pixels constituting the monitoring sensors 37 among the pixels are constructed to retrieve the second dose signal S4 according to the generated charge even while the normal pixels operate for storing. Examples of the monitoring sensors 37 are pixels of which a source and a drain of the TFT are short-circuited, pixels of which a photoconductor is directly connected to a signal line without a TFT for flowing of generated charge to the signal processing circuit irrespective of turn-on or turn-off of the TFT, or pixels of which a TFT is driven discretely from TFTs for the normal pixels for imaging.

In case the detection panel 35 changes over from the ready mode (repetition of the pixel reset) to the imaging mode (start of the storing), then the arithmetic unit 38 starts sampling the second dose signal S4. At each time of the sampling the second dose signal S4, the arithmetic unit 38 calculates an average of the second dose signal S4 from the monitoring sensors 37 within a radiation receiving area determined according to the body part. Note that a maximum value, mode value, total or the like may be used for a representative in place of the average.

The arithmetic unit 38 calibrates the second dose signal S4 for a value corresponding to the first dose signal from the ionization chamber 30 for the purpose of receivability in the AEC interface 29. Specifically, the arithmetic unit 38 multiplies the second dose signal S4 by a coefficient according to an output level of the first and second dose signals in a state of irradiation of X-rays without presence of the object H. For example, let 1 be an output level of the first dose signal in a state of irradiation of X-rays without presence of the object H. Let 10 be an output level of the second dose signal S4 in the state of irradiation of X-rays without presence of the object H. Then the second dose signal S4 is multiplied by 0.1. Also, it is possible to calculate the coefficient for the second dose signal S4 according to various parameters, which can be sensitivity of the ionization chamber 30 and the monitoring sensors 37 to X-rays, a distance between the X-ray source 10 and the ionization chamber 30, a distance between the X-ray source 10 and the monitoring sensors 37 (the active pixel area 35a of the detection panel 35).

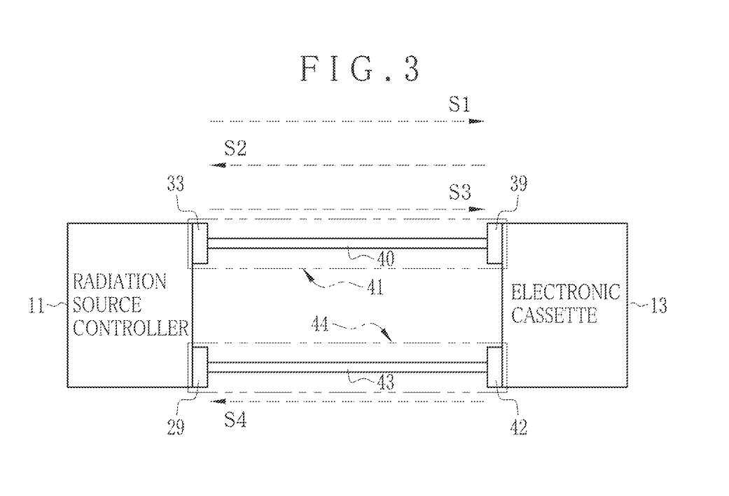

In FIG. 3, a sync communication interface 39 (I/F) or synchronization interface is coupled to the sync communication interface 33 of the radiation source controller 11 by a cable line 40 or wire line. A first communication path 41 or signal path is established by the sync communication interface 33, the sync communication interface 39 and the cable line 40. The sync communication interface 39, disposed downstream of the sync communication interface 33, receives a request signal S1 output by the sync communication interface 33 as an input to the controller 36. In response to the request signal S1, the controller 36 changes over the detection panel 35 from the pixel reset to the storing, and changes over from the ready mode to the imaging mode. At the same time, the controller 36 outputs an enable signal S2 to the sync communication interface 33 from the sync communication interface 39.

An AEC interface 42 (I/F) or monitor interface is connected to the AEC interface 29 of the radiation source controller 11 by a cable line 43 or wire line. The AEC interface 42 outputs the second dose signal S4 from the arithmetic unit 38 to the AEC interface 29. Thus, a second communication path 44 or signal path is established by the AEC interfaces 29 and 42 and the cable line 43.

A communication interface 45 (I/F) is coupled to the console unit 14 in a form communicable wirelessly or in a wired manner. Examples of information transmitted through the communication interface 45 are X-ray image data output by the detection panel 35, imaging condition set by the console unit 14, and the like.

A portable housing in a substantially quadrilateral form of a small thickness contains the detection panel 35 and the controller 36. The housing also contains a battery and an antenna in addition to the detection panel 35. The battery (secondary cell) supplies elements of the electronic cassette 13 with power at a predetermined voltage. The antenna operates for wireless communication with the console unit 14 for data such as an X-ray image and the like.

The housing has a size according to the international standards ISO 4090:2001 in a manner similar to the film cassette and IP cassette. The electronic cassette 13 is removably set on any one of the cassette holders 15a and 16a of the imaging stations 15 and 16 well-known in combination with the film cassette and IP cassette to oppose the active pixel area 35a of the detection panel 35 to the X-ray source 10. A moving mechanism moves the X-ray source 10 suitably for one of the imaging stations 15 and 16 for use. Note that it is possible to use the electronic cassette 13 in different manners without the use of the imaging stations 15 and 16. For example, the electronic cassette 13 can be placed on a bed or table where the body H lies. The patient (body H) can manually hold the electronic cassette 13 for imaging. Furthermore, the electronic cassette 13 can have a size different from that according to the international standards ISO 4090:2001.

A user input interface 50 or input device is connected to the console unit 14, such as a keyboard and the like. The console unit 14 controls the electronic cassette 13 according to an input generated by manipulation of a physician or operator for the user input interface 50. A monitor display panel 51 of the console unit 14 displays an X-ray image sent by the electronic cassette 13 through the communication interface 45. Also, a storage medium 52 in the console unit 14 stores the X-ray image, such as a memory, hard disk device and the like. Also, an image server or the like as a data storage stores the X-ray image in connection with the console unit 14 in a network.

The console unit 14 receives an input of an imaging request having information of sex and age of the body H, and a body part and purpose of imaging. The monitor display panel 51 is driven to display the imaging request. An external managing system (not shown) for managing object information and imaging information of radiographic imaging is connected to the console unit 14 and inputs the imaging request. Examples of the external managing system are the HIS (hospital information system) and the RIS (radiology information system). Also, it is also possible for a physician or operator manually to input the imaging request. Examples of the body parts in the imaging request are a head, chest, abdomen, hand, fingers and the like. Also, a viewing direction is included in the imaging request, such as a front direction, lateral direction, diagonal direction, PA (posteroanterior direction), AP (anteroposterior direction), and the like. The physician or operator checks the imaging request on the monitor display panel 51, and manually inputs an imaging condition with the user input interface 50 by viewing the input menu on the monitor display panel 51.

Information of imaging conditions is previously stored in the console unit 14 for each of body parts. Each imaging condition includes a tube voltage, tube current, radiation receiving area and the like. The information of the imaging conditions is stored in the storage medium 52. One of imaging conditions is read from the storage medium 52 according to input information of a body part with the user input interface 50. The electronic cassette 13 is supplied with the selected imaging condition through a communication interface 53 (I/F). For the radiation source controller 11, a physician or operator views the content of the imaging condition of the console unit 14, and manually inputs the imaging condition in the same manner.

The control unit 26 in the radiation source controller 11 checks whether the cumulative dose of X-rays to the active pixel area 35a has come up to the target dose according to the first or second dose signal input to the AEC interface 29. The control unit 26 accumulates the first or second dose signal, compares the cumulative value (cumulative dose) of the accumulation with a predetermined stop threshold (target dose), and performs the checking of the dose.

In case the control unit 26 judges that the cumulative value becomes higher than the stop threshold and the cumulative dose of the X-rays comes up to the target does, then the control unit 26 stops the high voltage source 25 from powering the X-ray tube 20. The X-ray irradiation is stopped. Note that a level of the dose signal may be considerably low under influence of implant. Then the control unit 26 can interrupt the X-ray irradiation by judging occurrence of abnormality.

Assuming that the electronic cassette is selected for use, the control unit 26 outputs an end flag S3 through the sync communication interface 33 to notify the end of the X-ray irradiation at the same time as stopping the X-ray irradiation. The end flag S3 from the sync communication interface 33 is input to the controller 36 through the sync communication interface 39. The controller 36 changes over the detection panel 35 from the storing to the readout upon receiving the end flag S3 from the sync communication interface 39.

Furthermore, the control unit 26 stops the X-ray irradiation in case the countdown timer measures the irradiation time predetermined with the touchscreen device 28, and in case the operation signal from the switch interface 32 discontinues by interruption of the full depression of the radiation switch 12.

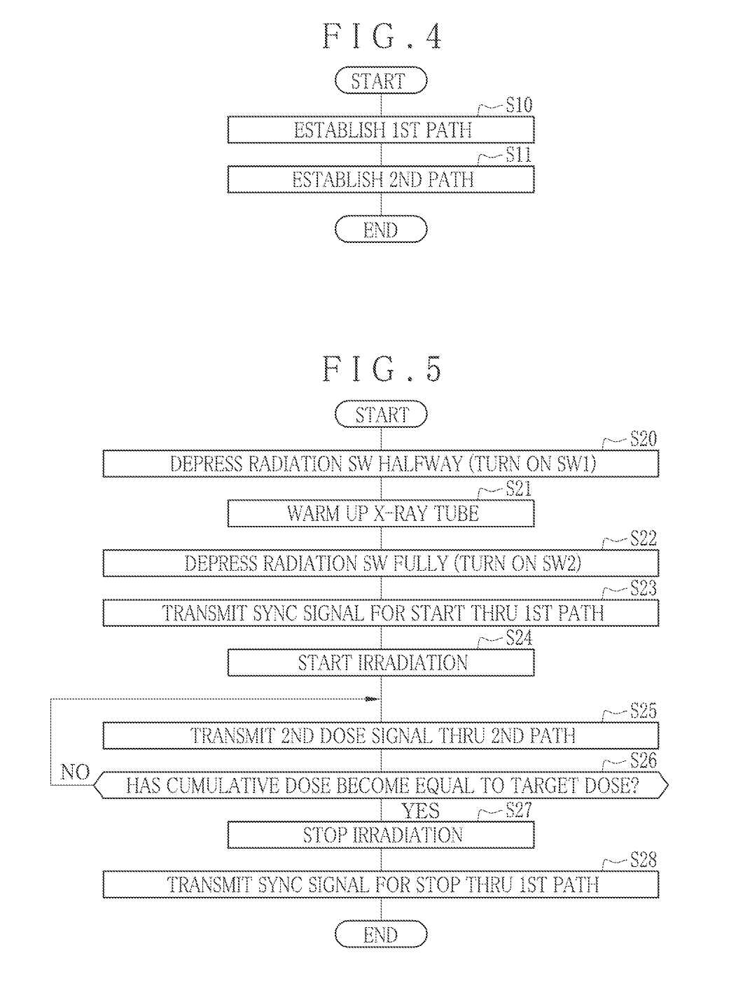

The operation of the imaging by use of the electronic cassette 13 in the X-ray imaging system 2 is now described by referring to FIGS. 4 and 5.

At first, the sync communication interface 33 of the radiation source controller 11 is connected to the sync communication interface 39 of the electronic cassette 13 by the cable line 40 in FIG. 4, to establish the first communication path 41 in the step S10. Also, the AEC interface 29 of the radiation source controller 11 is connected to the AEC interface 42 of the electronic cassette 13 by the cable line 43, to establish the second communication path 44 in the step S11.

In case the preparatory operation is completed, the body H is positioned on one of the imaging stations 15 and 16 for the standing or horizontal orientation. A height and horizontal position of the electronic cassette 13 are adjusted to target one of body parts of the body H. The height, horizontal position, size of radiation receiving area of the X-ray source 10 are adjusted according to the position of the electronic cassette 13 and a size of the body part. Then an imaging condition is manually set in the radiation source controller 11 and the console unit 14. Information of the imaging condition of the console unit 14 is transmitted to the electronic cassette 13.

In FIG. 5, the radiation switch 12 depressed halfway to turn on the switch SW1 by the physician or operator after completing preparation for the imaging, in the step S20. The radiation source controller 11 outputs a warmup signal for starting warmup to the high voltage source 25. The high voltage source 25 starts powering the X-ray tube 20 to warm up the X-ray tube 20 in the step S21.

After halfway depressing the radiation switch 12, the physician or operator estimates time for the warmup, and then fully depresses the radiation switch 12 (turns on the switch SW2) in the step S22. The request signal S1 and the enable signal S2 (corresponding to sync signals or monitoring signal) are transmitted through the first communication path 41 between the radiation source controller 11 and the electronic cassette 13 in the step S23. The detection panel 35 in the electronic cassette 13 is changed over from the pixel reset to the storing. The arithmetic unit 38 starts sampling the second dose signal S4.

The radiation source controller 11 upon receiving the enable signal S2 from the electronic cassette 13 generates a drive signal, so that the X-ray tube 20 emits X-rays in the step S24.

Upon starting the irradiation of X-rays, the second dose signal S4 according to measured dose is output by the monitoring sensors 37. The second dose signal S4 is sent to the arithmetic unit 38. The arithmetic unit 38 calculates an average of the dose signal from the monitoring sensors 37 located within the radiation receiving area according to information of the radiation receiving area from the console unit 14. The average is calibrated by the arithmetic unit 38 into a level corresponding to the first dose signal, before a signal of the average is transmitted to the radiation source controller 11 through the second communication path 44 in the step S25.

The radiation source controller 11 determines a cumulative value of the average of a second dose signal S4 (corresponding to an AEC signal or monitoring signal) successively input by the electronic cassette 13. The cumulative value is compared with the stop threshold of the irradiation. Those steps are repeated at each time of the sampling of the second dose signal S4 (no in the step S26).

In case the cumulative value comes up to the stop threshold (yes in the step S26), then the radiation source controller 11 judges that reach of the cumulative dose to the target dose of X-rays, stops the high voltage source 25 from powering the X-ray tube 20, and stops irradiation of X-rays of the X-ray source 10 in the step S27. Also, the radiation source controller 11 outputs the end flag S3 to the electronic cassette 13 through the first communication path 41 in the step S28.

In the electronic cassette 13, the detection panel 35 is changed over from the storing to the readout upon receiving the end flag S3 from the radiation source controller 11, to output X-ray image data. After the readout, the detection panel 35 returns to the ready mode for the pixel reset. The X-ray image data from the detection panel 35 is processed in functions of the image processing, and transmitted to the console unit 14, so that the monitor display panel 51 is driven to display an image of the image data for diagnosis. Then one session of X-ray imaging is completed.

Assuming that a film cassette or IP cassette or an electronic cassette without an AEC is used for imaging, the ionization chamber 30 is connected to the AEC interface 29. The AEC is performed in the control unit 26 according to the first dose signal from the ionization chamber 30. Assuming that no AEC is performed, irradiation of X-rays is stopped upon lapse of the irradiation time set by the touchscreen device 28 according to the measurement in the countdown timer.

As described heretofore, the AEC interface 29 is provided in nearly every one of various types of the radiation source controller 11. The radiation source controller 11 checks whether the cumulative dose has come up to a target dose according to the first dose signal from the ionization chamber 30 through the AEC interface 29. Thus, it is possible to use the electronic cassette 13 with the AEC function without modifying the radiation source controller 11 by communication of the second dose signal S4 through the second communication path 44 established by the AEC interface 29. Even a hospital facility which has not decided financially to introduce the electronic cassette 13 can introduce the electronic cassette 13 owing to the use of the radiation source controller 11 of the same structure, so that a manufacturer or dealer of medical instruments can promote the electronic cassette 13 widely and easily with a relatively low cost. Also, it is also possible to utilize normal types of a film cassette, IP cassette, electronic cassette without an AEC function and the like.

The ionization chamber 30 is a device which has been well-known technically for use. The AEC interface 29 is provided in nearly every one of various types of the radiation source controller 11. The standards of the AEC interface 29 are limited without complicated variety. Consequently, regularization of the form of the second dose signal is effective in compatibility for various types of the X-ray generating apparatus 2a provided by plural manufacturers, to obtain good advantages.

In the above embodiment, the radiation source controller 11 has the sync communication interface 33. However, some type of a radiation source controller does not have the sync communication interface 33 in an X-ray generating apparatus for a film cassette or IP cassette without compatibility to the radiation source controller.

Note that an anti-scatter grid may be used in the X-ray imaging system for eliminating stripes of scattering created in transmission of X-rays through the body H. An example of the anti-scatter grid includes a thin plate, having radio-transparent segments, arranged in an array in a row direction of the pixels, to extend in a strip form in a columnar direction of the pixels, and formed from a radio-transparent layer transmitting X-rays, and radiopaque segments, arranged in a strip form and alternately with the radio-transparent segments in the row direction of the pixels, and formed from a radiopaque layer blocking X-rays. The anti-scatter grid is positioned between the body H and the electronic cassette, and opposed to a surface of the electronic cassette on the entrance side of X-rays.

The X-ray imaging system with the anti-scatter grid can have a bookie device, which can be disposed on an imaging station, for moving the anti-scatter grid from the start of the X-ray irradiation until the stop, for making stripes of the anti-scatter grid inconspicuous with radio-transparent and radiopaque segments. A control interface may be provided in a radiation source controller for synchronization with the bookie device for the start and stop of the X-ray irradiation. For this structure of the radiation source controller, it is possible to connect the electronic cassette to the control interface for the bookie device, and to perform synchronization of the start from the pixel reset to the storing of the detection panel 35 according to the signal from the control interface for the bookie device, or synchronization of the stop from the storing to the readout. In short, it is possible to use the control interface for the bookie device by way of a sync communication interface.

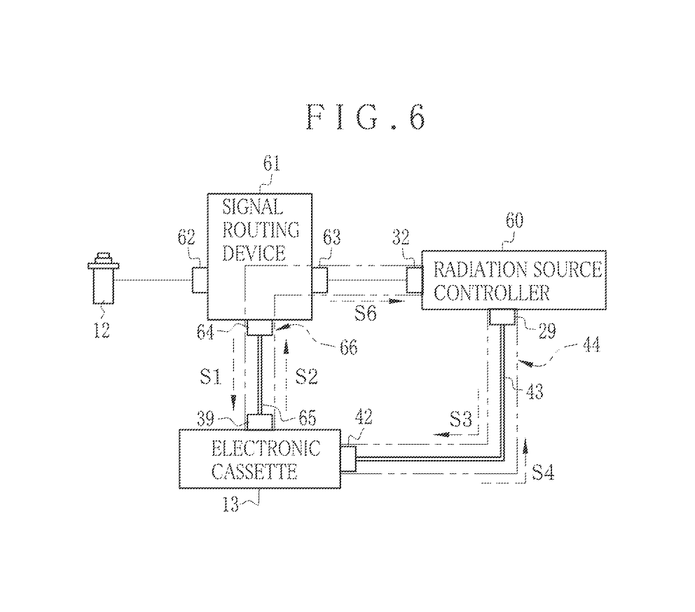

An X-ray imaging system may not have a control interface for the bookie device. Otherwise, it is likely that a control interface for a bookie device cannot be used because the control interface is coupled with a bookie device and is unavailable for other purposes. However, a construction of FIG. 6 is usable for solving such problems.

### Second Embodiment

In FIG. 6, another preferred radiation source controller 60 is illustrated. The radiation source controller 11 of the first embodiment is repeated but with a difference in not having the sync communication interface 33. Elements similar to those of the above embodiment are designated with identical reference numerals. A signal routing device 61 or signal relay device is connected between the radiation switch 12 and the switch interface 32 for communication in synchronism with the electronic cassette 13 in place of the radiation source controller 60.

The signal routing device 61 includes a first interface port 62 (I/F), a second interface port 63 (I/F) and a third interface port 64 (I/F), or connection interfaces. The radiation switch 12 is coupled with the first interface port 62. The switch interface 32 is coupled with the second interface port 63. The sync communication interface 39 of the electronic cassette 13 is coupled with the third interface port 64 by a cable line 65 or wire line.

The signal routing device 61 outputs an operation signal of the halfway depression of the radiation switch 12 from the first interface port 62 to the radiation source controller 60 through the second interface port 63. The signal routing device 61 generates a request signal S1 upon receiving an operation signal of the full depression of the radiation switch 12 with the first interface port 62, and outputs the request signal S1 to the electronic cassette 13 through the third interface port 64. Also, the signal routing device 61 receives the enable signal S2 from the electronic cassette 13 through the cable line 65 and the third interface port 64. In short, a first communication path 66 or signal path is established by the sync communication interface 39, the third interface port 64, the cable line 65, the second interface port 63 and the switch interface 32.

The signal routing device 61 upon receiving the enable signal S2 from the electronic cassette 13 generates a pseudo signal S6 corresponding to the operation signal of the full depression of the radiation switch 12. The pseudo signal S6 is transmitted from the second interface port 63 to the radiation source controller 60. The radiation source controller 60 upon receiving the pseudo signal S6 transmits an instruction signal to the high voltage source 25 in a similar manner to occurrence of the operation signal of the full depression from the radiation switch 12 upon direct connection of the radiation switch 12 to the switch interface 32. Irradiation of X-rays is started. Thus, it is possible with the signal routing device 61 to image an object by use of the X-ray generating apparatus without synchronization with the electronic cassette. Then the electronic cassette 13 receives the end flag S3 from the radiation source controller 11 through the second communication path 44.

### Third Embodiment

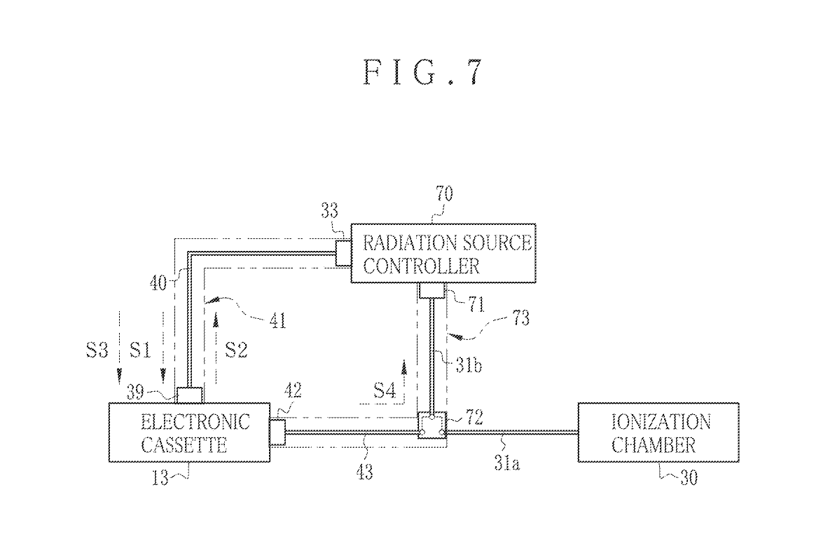

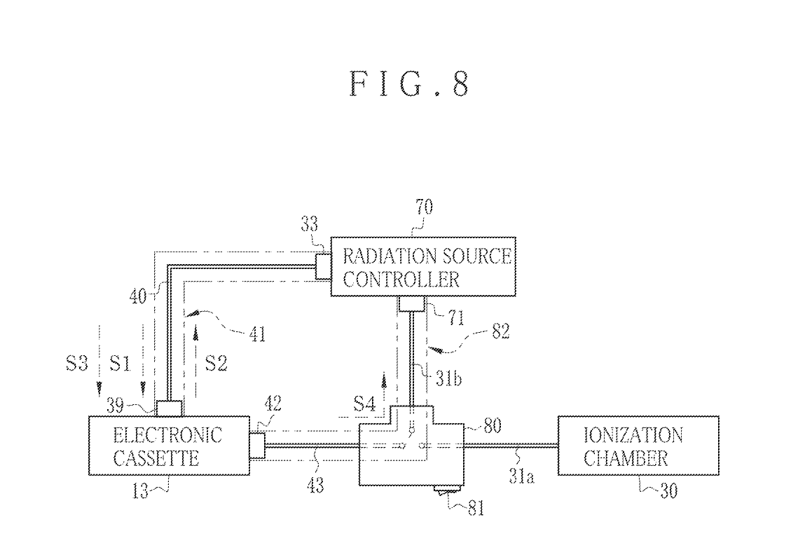

In the above embodiments, the ionization chamber 30 is removable from the AEC interface 29, and removed for the use of the electronic cassette 13. In contrast, another preferred radiation source controller is provided, in which the ionization chamber 30 is incorporated without removable structure. See FIGS. 7 and 8.

In a radiation source controller 70 (source driver) of FIGS. 7 and 8, the radiation source controller 11 of the first embodiment is repeated but with a difference in that the cable line 31 is fixedly connected to an AEC interface 71 (I/F) and the ionization chamber 30, which is not removable. Elements similar to those of the embodiments are designated with identical numerals.

In FIG. 7, the cable line 31 is formed with branches of signal lines 31a and 31b. There is a terminal board 72 having input terminals and an output terminal as a branch connector (multi-port connector). The signal line 31a of the side of the ionization chamber 30 and the cable line 43 are connected to input terminals (second and third terminals) of the terminal board 72. The signal line 31b of the AEC interface 71 is connected to the output terminal (first terminal) of the terminal board 72. Thus, a second communication path 73 or signal path is established by the serial connection of the cable lines 31 and 43. Note that a selected one of the AEC of the electronic cassette 13 and the ionization chamber 30 not for use is turned off for the power, or a signal line of the selected one is disconnected from the terminal board 72. This is because simultaneous operation of the AEC of the electronic cassette 13 and the ionization chamber 30 may cause crosstalk between the first and second dose signals so that the radiation source controller 70 generates an erroneous result.

In FIG. 8, a selector 80 is connected to the signal lines 31a and 31b and the cable line 43 as a branch connector (multi-port connector) in place of the terminal board 72. A selection switch 81 is provided on the outside of the selector 80, and changes over the connection of the signal line 31b between the signal line 31a and the cable line 43. For use of the AEC of the electronic cassette 13, the selector 80 selects the side of the cable line 43. A second communication path 82 or signal path is established and inputs the second dose signal S4 to the AEC interface 71. Assuming that the ionization chamber 30 is used, the selector 80 selects the side of the signal line 31a, so that the first dose signal is input to the AEC interface 71. Therefore, the second dose signal S4 is prevented from being input to the radiation source controller 70 simultaneously with the first dose signal in contrast with the feature of FIG. 7. Erroneous evaluation in the radiation source controller 70 is prevented.

### Fourth Embodiment

In the above embodiments, the first and second communication paths are in a wired structure with signal lines.

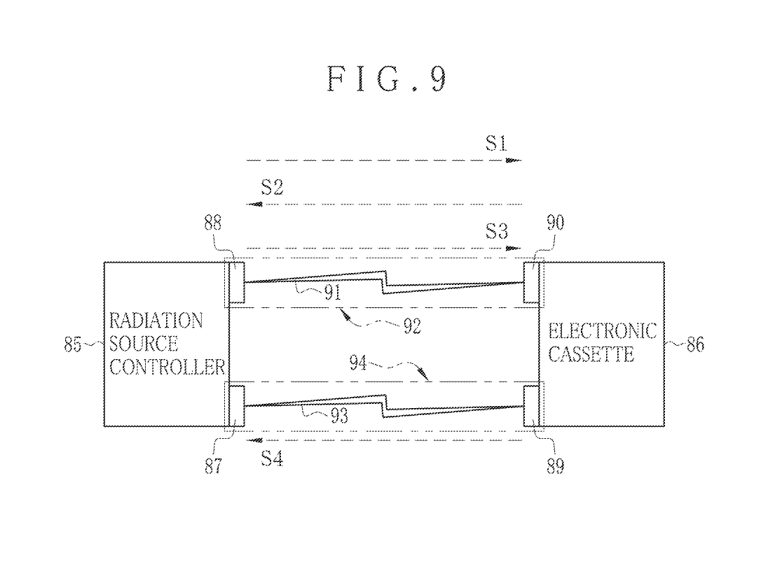

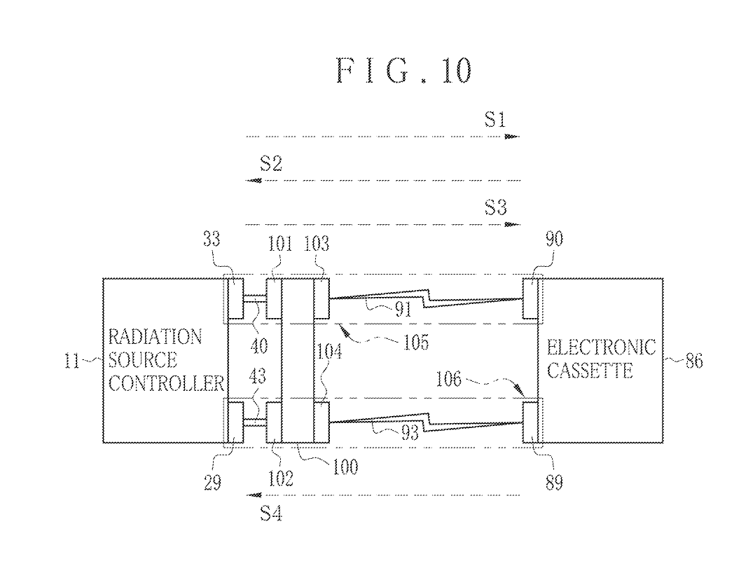

In contrast, FIGS. 9 and 10 illustrate a preferred embodiment in which communication paths are at least partially wireless.

In FIG. 9, a radiation source controller 85 includes an AEC interface 87 (I/F) and a sync communication interface 88 (I/F) for wirelessly transmitting signals. An electronic cassette 86 includes an AEC interface 89 (I/F) and a sync communication interface 90 (I/F) for wireless communication with respectively the AEC interface 87 and the sync communication interface 88.

A first radio wave 91 is transmitted between the sync communication interfaces 88 and 90 for the request signal S1, enable signal S2 and end flag S3 in a similar manner to a path between the sync communication interfaces 33 and 39 in the first embodiment. In short, a first communication path 92 or signal path is established by the sync communication interfaces 88 and 90 and the first radio wave 91. Also, a second radio wave 93 is transmitted between the AEC interfaces 87 and 89 for the second dose signal S4 in a similar manner to a path between the AEC interfaces 29 and 42 in the first embodiment. A second communication path 94 or signal path is established by the AEC interfaces 87 and 89 and the second radio wave 93.

In FIG. 10, a signal routing device 100 or signal relay device is connected between the electronic cassette 86 and the radiation source controller 11 of the first embodiment. The signal routing device 100 includes a first interface port 101 (I/F), a second interface port 102 (I/F), a third interface port 103 (I/F) and a fourth interface port 104 (I/F), or connection interfaces. The first interface port 101 is coupled to the sync communication interface 33 of the radiation source controller 11 by the cable line 40. The second interface port 102 is coupled to the AEC interface 29 by the cable line 43. The third interface port 103 is coupled to the sync communication interface 90 of the electronic cassette 86 by the first radio wave 91. The fourth interface port 104 is coupled to the AEC interface 89 of the electronic cassette 86 by the second radio wave 93. The signal routing device 100 is disposed near to the radiation source controller 11. Most of its communication path is constituted by the first and second radio waves 91 and 93. A first communication path 105 and a second communication path 106 or signal paths are established by the AEC interface 29, the sync communication interface 33, the AEC interface 89, the sync communication interface 90, the interface ports 101-104, the cable lines 40 and 43 and the first and second radio waves 91 and 93.

Consequently, component apparatuses in the X-ray imaging system can be arranged with a high degree of freedom in the layout without considering disposition of the cable lines, because of the wireless connection of the entirety or part of the first and second communication paths. As the electronic cassette 86 is wireless, handlability of the electronic cassette 86 can be made high unlike a cassette with normal handlability in which two communication paths are wired. In FIG. 10, the use of the signal routing device 100 makes it possible to use the electronic cassette 86 of the wireless communication even with the radiation source controller 11 without compatibility to the wireless communication. Note that the first and second radio waves 91 and 93 have frequencies different from one another for the purpose of preventing crosstalk. Furthermore, it is possible to utilize optical communication of infrared radiation or the like in place of radio waves.

### Fifth Embodiment

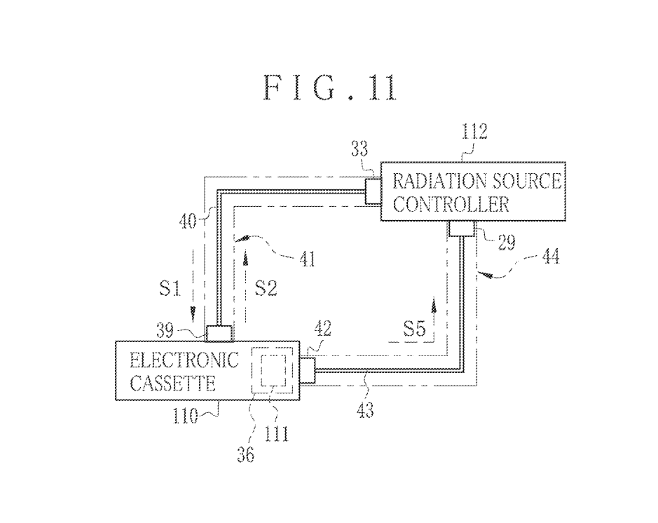

In the above embodiments, the reach of the cumulative dose to the target dose is detected in the radiation source controller. In FIG. 11, another preferred electronic cassette detects the reach of the cumulative dose to the target dose.

In FIG. 11, an electronic cassette 110 is an X-ray imaging apparatus with the AEC inclusive of an AEC signal output device in a manner similar to the electronic cassette 13. The electronic cassette 110 includes the monitoring sensors 37 and the controller 36 with the arithmetic unit 38. Furthermore, an evaluator 111 is provided in the controller 36 for detecting a reach of the cumulative dose of X-rays to a target dose. The evaluator 111 accumulates the second dose signal S4 obtained at each time of sampling in the arithmetic unit 38 downstream of the monitoring sensors 37, and obtains a cumulative value byway of a cumulative dose of X-rays. Then the evaluator 111 compares the cumulative dose with the target dose, and checks whether the cumulative dose becomes equal to the target dose. There is a radiation source controller 112, to which the controller 36 outputs a stop signal S5 (corresponding to an AEC signal) of irradiation assuming that the evaluator 111 judges that the cumulative dose comes up to the target dose. The radiation source controller 112 receives a stop signal S5 from the electronic cassette 110, and stops the X-ray irradiation of the X-ray source 10. In the electronic cassette 110, the AEC signal output device is constituted by the monitoring sensors 37 and the controller 36 which has the arithmetic unit 38 and the evaluator 111.

The AEC signal from the electronic cassette is converted into a form receivable by the AEC interface of the radiation source controller. The arithmetic unit 38 in the first embodiment operates for this purpose. However, other structure can perform the conversion as will be described next.

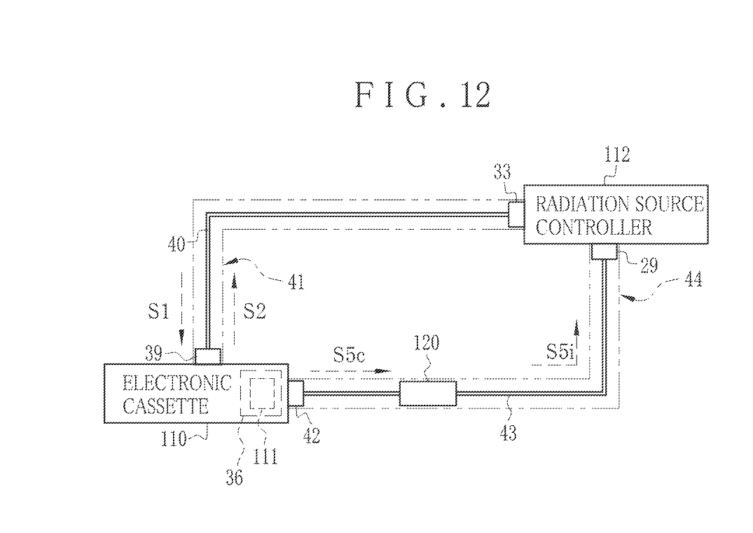

In FIG. 12, a variant of the embodiment of FIG. 11 is illustrated. Stop signals from plural devices may differ in their signal form. For example, a stop signal S5i from the ionization chamber 30 is a high level signal of +5 V. A stop signal S5c from the electronic cassette 110 is a low level signal of −10 V. For this difference, a signal converter 120 is provided in the cable line 43 for converting the stop signal S5c to the stop signal S5i. Thus, the radiation of X-rays can be properly stopped by the radiation source controller 112 no matter which of the ionization chamber 30 and the electronic cassette 110 is used. Note that the signal converter 120 can be incorporated in the electronic cassette 110 in the manner of the arithmetic unit 38 of the first embodiment. Furthermore, the arithmetic unit 38 can be a component separate from the electronic cassette 13.

In the drawing, the signal converter 120 is provided in the cable line 43. However, the signal converter 120 can have a plug-in structure with terminals or contacts for direct connection to either one of the AEC interfaces 29 and 42. Also, the feature of FIG. 12 can be combined with that of FIG. 10, so the signal routing device 100 can perform the signal conversion of the signal converter 120.

Furthermore, a combination of the electronic cassette and the radiation source controller can be constructed so that the radiation source controller can receive the dose signal as an AEC signal but cannot receive a stop signal. The AEC signal from the electronic cassette should be converted into a form receivable by the AEC interface of the radiation source controller. To this end, an output function is provided in the electronic cassette for outputting a dummy dose signal from the electronic cassette to the radiation source controller continuously until an evaluator in the electronic cassette detects a reach of the cumulative dose to the target dose. Upon the detection of the reach of the cumulative dose to the target dose in the evaluator in the electronic cassette, a signal is output from the electronic cassette to the radiation source controller at a level corresponding to a dose signal of possibility of detecting the reach of the cumulative dose to the target dose in an evaluator in the radiation source controller.

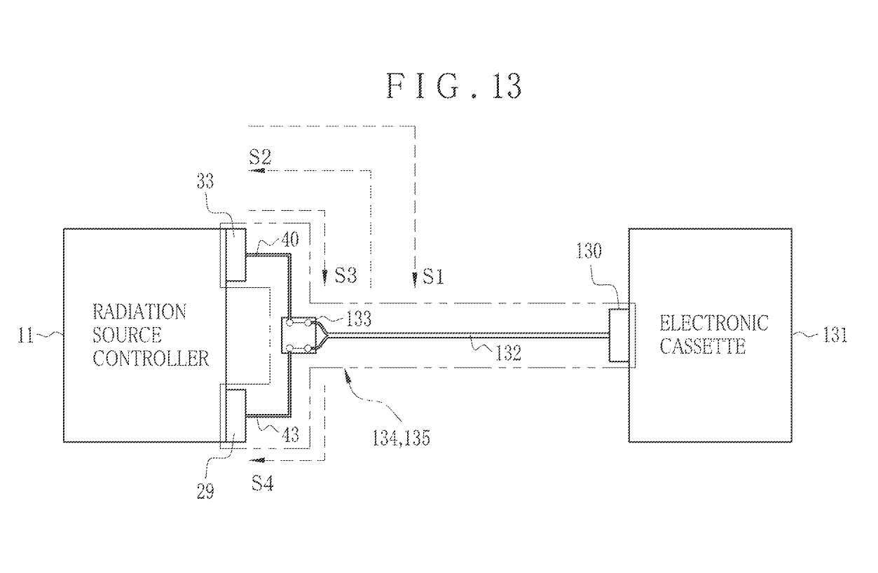

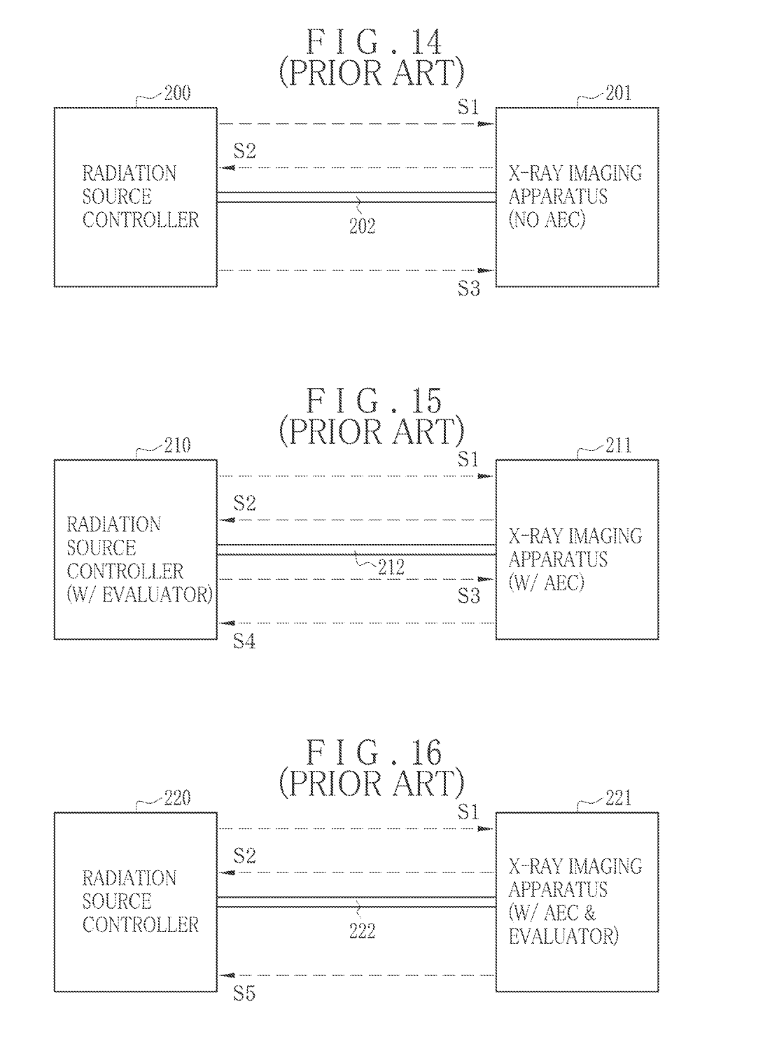

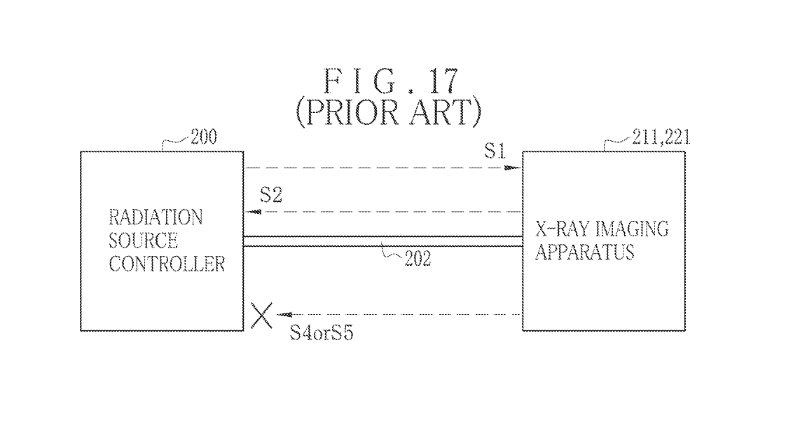

In the above embodiments, the AEC interface (I/F) is discrete from the sync communication interface (I/F) in the electronic cassette to establish the first and second communication paths. In FIG. 13, a variant of the embodiment of FIG. 3 is illustrated. An electronic cassette 131 has one common interface 130 (I/F) having functions of the AEC interface (I/F) and the sync communication interface (I/F). A cable line 132 or wire line (third cable line) is provided as a single element in contrast with the cable lines 40 and 43. A first end of the cable line 132 is connected to the common interface 130. A second end of the cable line 132 has the branches of the cable lines 40 and 43 (first and second cable lines), which are connected to a first one of terminals of a terminal board 133. The cable lines 40 and 43 from the radiation source controller 11 are connected to the remaining terminals of the terminal board 133. Thus, first and second communication paths 134 and 135 or signal paths are established in a partially common form. The terminal board 133 is disposed near to the radiation source controller 11. A large part of the communication paths is constituted by the cable line 132. Thus, the number of the parts of the interfaces can be reduced in the electronic cassette. The cable line 132 has the branches of the cable lines 40 and 43 in the electronic cassette 131, for connection to the processing devices for the sync communication and AEC. In the radiation source controller 60 without the sync communication interface in FIG. 6, the cable line 40 is connected to the third interface port 64 of the signal routing device 61.

Also, two or more features of the above embodiments may be combined with one another. For example, a part or entirety of the second communication path in FIGS. 7 and 8 can be wireless.

Note that it is possible to monitor a current in a bias line connected to particular pixels of the detection panel so as to detect a dose, by utilizing a flow of the current according to charge generated by the pixel in the bias line for supplying bias voltage to the pixel. A dose sensor is constituted by the pixel for monitoring the current of the bias line. Furthermore, it is possible to monitor a leak current flowing out of the pixel. A dose sensor is constituted by the pixel for monitoring the leak current. Also, a discrete dose sensor can be provided in an active pixel area in a manner of discretely generating an output with a difference from pixels.

In the above embodiments, the electronic cassette is discrete from the console unit. However, a component of the console unit including a display panel can be incorporated in an electronic cassette. Also, a specialized imaging control unit can be connected between the electronic cassette and the console unit.

Note that the switch interface 32 and the first interface port 62 in the above embodiments can be a connector simply having a terminal or contact.

The terminal board 72 and the selector 80 are connected to the three devices by the signal lines 31a and 31b and the cable line 43. However, any one of the signal lines 31a and 31b and the cable line 43 can be replaced with terminals or contacts of a plug-in structure of a direct connection.

In the above embodiments, the sensor panel has the TFT. However, a sensor panel may be a CMOS type. In the above embodiments, the electronic cassette is portable. However, an X-ray imaging apparatus of the invention can be an installed type for an imaging stand without portability. In the above embodiments, the radiation is X-rays. However, radiation in the radiographic imaging may be gamma rays or the like.

Although the present invention has been fully described by way of the preferred embodiments thereof with reference to the accompanying drawings, various changes and modifications will be apparent to those having skill in this field. Therefore, unless otherwise these changes and modifications depart from the scope of the present invention, they should be construed as included therein.

What is claimed is:

1. A communication control method for a radiographic imaging system including a radiation generating apparatus, having a radiation source for emitting radiation, and a radiation source controller for controlling said radiation source, and a radiographic imaging apparatus, having a detection panel for receiving said radiation transmitted through an object and converting a radiation image of said object into an electric signal, and an AEC signal output device for detecting a dose of said radiation and outputting an AEC signal for exposure control of said radiation image, said communication control method comprising steps of:
transmitting a sync signal through a first communication path between said radiation source controller and said radiographic imaging apparatus, for driving said detection panel in synchronism with a start of said irradiation of said radiation;
transmitting said AEC signal from said radiographic imaging apparatus to said radiation source controller through a second communication path between said radiation source controller and said radiographic imaging apparatus, by use of an AEC interface provided in said radiation source controller for connection with an AEC signal output device discrete from said radiographic imaging apparatus.

2. A communication control method as defined in claim 1, wherein said AEC signal is transmitted in a form receivable by said AEC interface.

3. A communication control method as defined in claim 2, wherein said radiographic imaging apparatus outputs said AEC signal in a form receivable by said AEC interface.

4. A communication control method as defined in claim 2, wherein said AEC signal from said radiographic imaging apparatus is converted by a signal converter in said second communication path into a form receivable by said AEC interface.

5. A communication control method as defined in claim 1, wherein said second communication path is at least partially wired with a cable line.

6. A communication control method as defined in claim 5, wherein a branch connector is provided in said cable line, and has first, second and third terminals;
said first terminal is coupled with said AEC interface, said second terminal is coupled with said radiographic imaging apparatus, and said third terminal is coupled with said AEC signal output device.

7. A communication control method as defined in claim 6, wherein said AEC signal output device and said radiographic imaging apparatus are changed over selectively in said branch connector for transmission of said AEC signal to said AEC interface.

8. A communication control method as defined in claim 1, wherein said second communication path is at least partially wireless.

9. A communication control method as defined in claim 8, wherein said AEC signal is output wirelessly by said radiographic imaging apparatus, then converted into a wired signal, and input to said AEC interface.

10. A communication control method as defined in claim 1, wherein said first communication path is established by a sync communication interface provided in said radiation source controller for transmitting said sync signal.

11. A communication control method as defined in claim 1, wherein a radiation switch is connected to said radiation source controller, and generates a signal for starting irradiation of said radiation, to establish said first communication path.

12. A communication control method as defined in claim 11, wherein said first communication path is established by a signal routing device connected to said radiation switch, said radiation generating apparatus and said radiographic imaging apparatus.

13. A communication control method as defined in claim 1, wherein said radiographic imaging apparatus includes:
a sync communication interface for establishing said first communication path;
a second AEC interface for establishing said second communication path.

14. A communication control method as defined in claim 1, wherein said radiographic imaging apparatus includes a common interface for communication of said sync signal and said AEC signal;
a first cable line is connected to said radiation source controller, for transmitting said sync signal;
a second cable line is connected to said AEC interface, for transmitting said AEC signal;
said first and second cable lines are coupled together and connected to a third cable line, said third cable line is connected to said common interface, for establishing said first and said second communication paths.

15. A communication control method as defined in claim 1, wherein said AEC signal output device includes a monitoring sensor for detecting said dose of said radiation;
said AEC signal is a dose signal from said monitoring sensor;
said radiation source controller includes an evaluator for checking whether a cumulative dose has become equal to a target dose according to said dose signal.

16. A communication control method as defined in claim 1, wherein said AEC signal output device includes:
a monitoring sensor for detecting said dose of said radiation;
an evaluator for checking whether a cumulative dose has become equal to a target dose according to a dose signal from said monitoring sensor;
assuming that said evaluator judges that said cumulative dose has become equal to said target dose, a stop signal is output for said AEC signal to stop said radiation source controller from irradiation.

17. A radiographic imaging system comprising:
a radiation generating apparatus, having a radiation source for emitting radiation, and a radiation source controller for controlling said radiation source;
a radiographic imaging apparatus, having a detection panel for receiving said radiation transmitted through an object and converting a radiation image of said object into an electric signal, and an AEC signal output device for detecting a dose of said radiation and outputting an AEC signal for exposure control of said radiation image;
a first communication path, established between said radiation source controller and said radiographic imaging apparatus, for transmitting a sync signal for driving said detection panel in synchronism with a start of said irradiation of said radiation;
a second communication path, established between said radiation source controller and said radiographic imaging apparatus by use of an AEC interface provided in said radiation source controller for connection with an AEC signal output device discrete from said radiographic imaging apparatus, for transmitting said AEC signal from said radiographic imaging apparatus to said radiation source controller.

18. A radiographic imaging apparatus comprising:
a detection panel for receiving radiation transmitted through an object after emission from a radiation source, and converting a radiation image of said object into an electric signal;
an AEC signal output device for detecting a dose of said radiation and outputting an AEC signal for exposure control of said radiation image;
a sync communication interface for establishing a first communication path for transmitting a sync signal to a radiation source controller for controlling said radiation source, for driving said detection panel in synchronism with a start of said irradiation of said radiation;
an AEC interface for coupling with a second AEC interface provided in said radiation source controller for connection with an AEC signal output device discrete from said AEC signal output device, and for establishing a second communication path for transmitting said AEC signal to said radiation source controller.
